SEQUENCE LISTING (5 sequences)

SEQ ID NO: 1 - DNA, 20 bp, Artificial sequence
  Synthetic primer
gaccaggagtggaggaaaca

SEQ ID NO: 2 - DNA, 20 bp, Artificial sequence
  Synthetic primer
cggccataatggtcactctt

SEQ ID NO: 3 - DNA, 20 bp, Artificial sequence
  Synthetic primer
cgtaccactggcatcgtgat

SEQ ID NO: 4 - DNA, 21 bp, Artificial sequence
  Synthetic primer
gtgttggcgtacaggtctttg

SEQ ID NO: 5 - protein, 4 aa, Artificial sequence
  Synthetic elastase substrate
AAPV

(19) United States
(12) Patent Application Publication
(10) Pub. No.: US 2009/0298797 A1
(43) Pub. Date: Dec. 3, 2009

(54) COMBINATION THERAPY FOR THE TREATMENT OF INFLUENZA

(75) Inventors: Bojian Zheng, Hong Kong (HK); Kowk-Yung Yuen, Hong Kong (HK)

Correspondence Address: Pabst Patent Group LLP, 1545 PEACHTREE STREET NE, SUITE 320, ATLANTA, GA, 30309, US

(73) Assignee: The University of Hong Kong

(21) Appl. No.: 12/469,319

(22) Filed: May 20, 2009

Related U.S. Application Data
(60) Provisional application No. 61/055,573, filed on May 23, 2008.

(51) Int. Cl. A61K 31/60; A61K 31/415; A61K 31/35; A61P 31/16

(52) U.S. Cl. 514/161; 514/406; 514/459

(57) ABSTRACT

Compositions and methods for treating one or more symptoms of influenza, preferably influenza due to infection with influenza A (H5N1) are provided. It has been discovered that administration of a combination of a neuraminidase inhibitor with two immunomodulators increases survivability in subjects 24, 48, or even 72 hours post infection compared to administration of the neuraminidase inhibitor alone. A preferred neuraminidase inhibitor is zanamivir. Preferred immunomodulators include, but are not limited to celecoxib and mesalazine. Another embodiment provides a method for treating influenza, preferably, influenza due to infection with avian influenza A (H5N1) by administering to subject infected with the influenza virus, an effective amount of a neuraminidase inhibitor to inhibit or reduce budding of the influenza virus from infected cells of the subject, and an effective amount of at least two immunomodulators effective to reduce or inhibit one or more symptoms of inflammation in the subject.

### CROSS-REFERENCE TO RELATED APPLICATIONS

This application claims priority to and benefit of U.S. Provisional Patent Application No. 61/055,573 filed on May 23, 2008 by Bojian Zheng and Kwok-Yung Yuen, and where permissible is incorporated by reference in its entirety.

### FIELD OF THE INVENTION

The invention is generally directed to compositions and methods for treating viral infections, in particular, influenza infection, especially avian influenza.

### BACKGROUND OF THE INVENTION

The mortality of patients suffering from pneumonia and multi-organ involvement due to influenza A/H5N1 virus has varied between 45% to 81% since the first report in 1997 (Yuen, K. Y., et al., Lancet 351:467-471 (1998); Beigel, J. H., et al., N Engl J Med 353:1374-1385 (2005)). Subsequent availability of the neuraminidase inhibitor, oseltamivir, has not reduced mortality. Oseltamivir is an antiviral drug that is used in the treatment and prophylaxis of both Influenzavirus A and Influenzavirus B. It acts as a transition-state analogue inhibitor of influenza neuraminidase, preventing progeny virions from emerging from infected cells. Oseltamivir was the first orally active neuraminidase inhibitor commercially developed. It is a prodrug, which is hydrolysed hepatically to the active metabolite, the free carboxylate of oseltamivir (GS4071). It is currently marketed under the trade name Tamiflu®.

The unsatisfactory outcome of patients treated with oseltamivir was attributed to either deficiencies in antiviral administration or the induction of a cytokine storm by the virus, leading to excessive local and systemic inflammatory response and multi-organ failure (Peiris, J. S., et al., Lancet 363:617-669 (2004)). The poor response to antivirals can also be the result of delayed initiation of treatment because of the non-specific initial manifestations of avian influenza, high initial viral load at the time of presentation, poor oral bioavailability of oseltamivir in the seriously ill, lack of intravenous preparations of neuraminidase inhibitors, and the emergence of resistance during therapy (Wong, S. S. and Yuen, K. Y., Chest 129:156-168 (2006); de Jong, M. D., et al., (2006) 12:1203-1207 (2006)). Attempts to use anti-inflammatory doses of corticosteroids to control excessive inflammation has been associated with severe side effects such as hyperglycemia or nosocomial infections without any improvement in survival (Carter, M. J., J Med Microbiol 56:875-883 (2007)). Moreover, TNF-α, IL-6 or CC chemokine ligand 2 knockout mice or steroid-treated wild-type mice did not have a significant survival advantage over wild type mice after viral challenge (Salomon, R., et al., Proc Natl Acad Sci USA 104:12479-12481 (2007)). This paradox can be explained if both a high viral load and the commensurate degree of excessive inflammation are as important in the pathogenesis and outcome of this highly lethal infection.

Currently, antiviral drugs, such as seltamivir, are effective for H5N1 avian flu patients if they are given the treatment within 48 hours after the onset. However, the mortality rate is over 70% if the patients receive the antiviral therapy more than 48 hour after onset. Although oseltamivir is highly effective in mouse models, the case-fatality rate remains very high in humans and delayed initiation of therapy appears to have a detrimental effect on survival. Thus, there is an urgent need to find an effective treatment strategy for influenza A/H5N1 virus infection in humans due to the substantial mortality.

Therefore, it is an object of the invention to provide compositions and methods for the treatment of viral infections, in particular influenza.

It is another object of the invention to provide compositions and methods for increasing survivability in patients infected with H5N1 avian flu.

### SUMMARY OF THE INVENTION

Compositions and methods for treating one or more symptoms of influenza, preferably influenza due to infection with avian influenza A (H5N1), are provided. It has been discovered that administration of a combination of a neuraminidase inhibitor with two immunomodulators increases survivability in subjects when administered 24, 48, or even 72 hours post infection compared to administration of the neuraminidase inhibitor alone. One embodiment provides an antiviral composition containing an effective amount of zanamivir, a pharmaceutically acceptable salt or prodrug thereof to inhibit or reduce influenza virus from budding from infected cells in a subject in combination with an effective amount of celecoxib and mesalazine or pharmaceutically acceptable salts or prodrugs thereof, to inhibit or reduce one or more symptoms of inflammation. Additional neuraminidase inhibitors include, but are not limited to, oseltamivir, peramivi, or pharmaceutically acceptable salts or prodrugs thereof. Other or additional anti-inflammatory agents can be used, for example, ligands of peroxisome proliferator-activated receptors alpha and gamma (PPARα or PPARγ) and other COX-2 inhibitors. Representative PPARα activators include, but are not limited to, fibrates such as gemfibrozil (e.g., Lopid®), bezafibrate (e.g., Bezalip®), ciprofibrate (e.g., Modalim®) clofibrate, renofibrate (e.g., TriCor®), or combinations thereof.

Another embodiment provides a method for treating influenza, preferably, influenza due to infection with avian influenza A (H5N1) by administering to an individual infected with the influenza virus, an effective amount of a neuraminidase inhibitor to inhibit or reduce budding of the influenza virus from infected cells of the subject, and an effective amount of at least two immunomodulators effective to reduce or inhibit one or more symptoms of inflammation in the subject.

### BRIEF DESCRIPTION OF THE DRAWINGS

FIG. 1A is a line graph of survival rates (percent) versus days post-challenge in mice (5 mice/group) treated with zanamivir (Z) (◯), celecoxib (C) (Δ), mesalazine (M) (□), gemfibrozil ( [symbol] ), celecoxib/mesalazine (C+M) (▴), celecoxib/gemfibrozil (C+G) (), or phosphate buffered saline (“PBS”) (control) (▪) at 4 hours post-challenge. FIG. 1B is a line graph of survival rates (percent) versus days post-challenge of the mice (10-15 mice/group) treated with zanamivir (Z) (◯), zanamivir/celecoxib (Z+C) (Δ), zanamivir/mesalazine (Z+M) (□), zanamivir/celecoxib/mesalazine (Z+C+M) (▪) or PBS (♦) at 48 hours post-challenge for 21 days. FIG. 1C is a line graph of weight (g+/−SD) versus days post-challenge of mice treated with zanamivir (Z) (◯), zanamivir/celecoxib (Z+C) (Δ), zanamivir/mesalazine (Z+M) (□) and zanamivir/celecoxib/mesalazine (Z+C+M) (▪) and PBS (♦) at 48 hours post-challenge for 21 days or until death.

FIG. 2A is a bar graph of viral titers versus days post-challenge in infected mice treated with zanamivir alone (Z), zanamivir/celecoxib/mesalazine (Z+C+M) or PBS, which was started at 48 hours post-challenge, as measured by TCID₅₀. The detection limit (undetectable) is 1:20. FIG. 2B is a bar graph of viral copies/100 β-actin versus days post-challenge in the mice from FIG. 2A.

FIGS. 3A-3P are bar graphs showing pg/ml of pro-inflammatory cytokines, chemokines, prostaglandins and albumin in tracheal-pulmonary lavage. Concentrations of IL-1 (FIGS. 3A, 3I), IL-6 (FIGS. 3B, 3J), IFN-γ (FIGS. 3C, K), TNF-α (FIGS. 3D, 3L), MIP-1 (FIGS. 3E, 3N), PGE2 (FIGS. 3F, 3M), leukotrienes (FIGS. 3G, 3O) and albumin (FIG. 3H) in tracheal-pulmonary lavage collected from mice treated with Z, Z+C+M, untreated control (PBS), or uninfected (normal) mice at indicated days were determined by ELISA, and compared between different groups. Lung injury was also assessed by measuring elastase activity in their tracheal-pulmonary lavage (FIG. 3P)

FIG. 4A is a bar graph of the number of CD3+/CD4+ T lymphocytes per 10,000 blood cells versus days post-challenge in mice treated with zanamivir alone (Z), zanamivir/celecoxib/mesalazine (Z+C+M) or PBS. FIG. 4B is a bar graph of the number of CD3+/CD8+ T lymphocytes per 10,000 blood cells versus days post-challenge in mice treated with zanamivir alone (Z), zanamivir/celecoxib/mesalazine (Z+C+M) or PBS. FIG. 4C is a graph of viral copies per 100 β-actin versus neutralizing antibody titer as determined by a cytopathic TCID₅₀ assay.

### DETAILED DESCRIPTION OF THE INVENTION

### I. Definitions

Unless otherwise defined, all technical and scientific terms used herein have the same meaning as commonly understood by one of ordinary skill in the art to which this invention pertains. All publications, patent applications, patents, and other references mentioned herein are incorporated by reference in their entirety. In case of conflict, the present specification, including definitions, will control. In addition, the materials, methods, and examples are illustrative only and not intended to be limiting.

The term “effective amount” or “therapeutically effective amount” means a dosage sufficient to provide treatment of influenza infection, particularly avian influenza A (H5N1) or to otherwise provide a desired pharmacologic and/or physiologic effect, for example, by reducing mortality or the severity of one or more symptoms. The precise dosage will vary according to a variety of factors such as subject-dependent variables (e.g., age, immune system health, etc.), and route of administration.

As used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “prodrug” refers to an active drug chemically transformed into a per se inactive derivative which, by virtue of chemical or enzymatic attack, is converted to the parent drug within the body before or after reaching the site of action. Prodrugs are frequently (though not necessarily) pharmacologically inactive until converted to the parent drug.

### II. Compositions

Compositions containing one or more neuraminidase inhibitors in combination with one or more immunomodulators are provided. A preferred composition has an effective amount of a neuraminidase inhibitor to inhibit or reduce influenza virus from budding from infected cells in a subject in combination with an effective amount of one or more, preferably at least two, anti-inflammatory agents, preferably non-steroidal anti-inflammatory agents to reduce inflammatory responses in the subject.

A. Neuraminidase Inhibitors

Neuraminidase inhibitors are a class of antiviral drugs targeted at the influenza viruses whose mode of action consists of blocking the function of the viral neuraminidase protein, thus preventing the virus from budding from the host cell (reproducing). Influenza neuraminidase exists as a mushroom-shaped projection on the surface of the influenza virus. It has a head consisting of four co-planar and roughly spherical subunits, and a hydrophobic region that is embedded within the interior of the virus' membrane. It includes a single polypeptide chain that is oriented in the opposite direction to the hemagglutinin antigen. The composition of the polypeptide is a single chain of six conserved polar amino acids, followed by hydrophilic, variable amino acids.

Neuraminidase has functions that aid in the efficiency of virus release from cells. Neuraminidase cleaves terminal neuraminic acid (also called sialic acid) residues from carbohydrate moieties on the surfaces of infected cells. This promotes the release of progeny viruses from infected cells. Neuraminidase also cleaves sialic acid residues from viral proteins, preventing aggregation of viruses. Administration of chemical inhibitors of neuraminidase is a treatment that limits the severity and spread of viral infections.

Neuraminidase also plays a role in the beginning of influenza pathogenesis by cleaving sialic acid from the host glycoprotein and allowing the virus to enter the host (T-phages, macrophages, etc.).

Representative neuraminidase inhibitors include, but are not limited to, oseltamivir, zanamivir and peramivir. Zanamivir is a neuraminidase inhibitor used in the treatment of and prophylaxis of both Influenza virus A and Influenzavirus B. Zanamivir was the first neuraminidase inhibitor commercially developed. Oseltamivir was the first orally active neuraminidase inhibitor commercially developed. It is a prodrug, which is hydrolysed hepatically to the active metabolite, the free carboxylate of oseltamivir (GS4071). Peramivir is an experimental antiviral drug still under development. These neuraminidase inhibitors are commercially available. Oseltamivir is sold under the tradename Tamiflu®. Zanamivir is sold under the tradename Relenza®. Peramivir is available from Biocryst Pharmaceuticals.

B. Immunomodulators

Preferred compositions for the treatment of influenza include one or more immunomodulators. Immunomodulators include immune suppressors or enhancers and anti-inflammatory agents. Preferred immunomodulators are anti-inflammatory agents. The anti-inflammatory agent can be non-steroidal, steroidal, or a combination thereof.

1. Non-Steroidal Anti-Inflammatory Agents

Preferred anti-inflammatory agents are non-steroidal anti-inflammatory (NSAID) agents. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

In one embodiment, immunomodulators are COX-2 inhibitors such as celecoxib and aminosalicylate drugs such as mesalazine and sulfasalazine. In a preferred embodiment, the disclosed composition contains an effective amount of zanamivir to inhibit or reduce influenza virus from budding from infected cells in a subject in combination with an effective amount of celecoxib and mesalazine to reduce inflammatory responses in the subject.

Celecoxib

Celecoxib is a non-steroidal anti-inflammatory drug (NSAID) used in the treatment of osteoarthritis, rheumatoid arthritis, acute pain, painful menstruation and menstrual symptoms, and to reduce numbers of colon and rectum polyps in patients with familial adenomatous polyposis. It has the brand name Celebrex®. Celecoxib is a highly selective COX-2 inhibitor and primarily inhibits the isoform of cyclooxygenase (inhibition of prostaglandin production), whereas traditional NSAIDs inhibit both COX-1 and COX-2. Celecoxib is approximately 7.6 times more selective for COX-2 inhibition over COX-1. In theory, this specificity allows celecoxib and other COX-2 inhibitors to reduce inflammation (and pain) while minimizing gastrointestinal adverse drug reactions (e.g., stomach ulcers) that are common with non-selective NSAIDs.

Mesalazine

Mesalazine, also known as mesalamine or 5-aminosalicylic acid (5-ASA), is an anti-inflammatory drug that is highly active in alimentary tract epithelial cells and is used to treat inflammation of the digestive tract (Crohn's disease) and mild to moderate ulcerative colitis. Mesalazine is a bowel-specific aminosalicylate drug that is metabolized in the gut and has its predominant actions there, thereby having few systemic side effects. As a derivative of salicylic acid, 5-ASA is also an antioxidant that traps free radicals, which are potentially damaging by-products of metabolism. 5-ASA is considered the active moiety of sulfasalazine, which is metabolized to it. Sulfasalazine (brand name Azulfidine® in the U.S., Salazopyrin in Europe) is a sulfa drug used primarily as an anti-inflammatory agent in the treatment of inflammatory bowel disease as well as for rheumatoid arthritis. It is not a pain killer.

Mesalazine and sulfasalazine have diverse effects on the immune system including inhibition of lipoxygenase and COX pathways, which decrease proinflammatory cytokines and eicosanoids, and therefore decrease the activation of inflammatory cells such as macrophages and neutrophils. In addition, sulfasalazine and 5-aminosalicylic acid inhibit NF-κB activation and promote the synthesis of phosphatidic acid. Both actions inhibit the potent stimulatory effects of ceramides on apoptosis.

Ligands of PPAR

PPAR are members of the nuclear receptor superfamily which affects the lipid and glucose metabolism, as well as modulation of inflammatory responses. PPAR-α and -γ ligands possess anti-inflammatory activities. PPARα activation is associated with inhibition of NF-KB, COX-2 activity, and production of pro-inflammatory cytokines such as IL-6 and TNF-α (Chinetti, G., et al., Inflamm Res 49:497-505 (2000)). Therefore, activation of the PPARα by gemfibrozil damp down the excessive inflammatory response. Budd et al. demonstrated that gemfibrozil improved survival of mice infected by influenza A/H2N2 virus from 26% (controls) to 52% (treated) (Budd, A., et al., Antimicrob Agents Chemother 51:2965-2968 (2007)). Representative PPAR ligands include, but are not limited to, fibrates. Exemplary fibrates include gemfibrozil (e.g., Lopid®), bezafibrate (e.g., Bezalip®), ciprofibrate (e.g., Modalim®). clofibrate, renofibrate (e.g., TriCor®), or combinations thereof.

2. Steroidal Anti-Inflammatory Agents

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, predisone, beclomethasone dipropionate, triamcinolone, and mixtures thereof.

The one or more active agents can be administered as the free acid or base or as a pharmaceutically acceptable acid addition or base addition salt.

Examples of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

C. Pharmaceutically Acceptable Salts

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

D. Formulations

Pharmaceutical compositions including as the active agents neuraminidase inhibitors in combination with immunomodulators are provided. The pharmaceutical compositions may be for administration by oral, parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in unit dosage forms appropriate for each route of administration. The preferred route is oral.

1. Formulations for Enteral Administration

In a preferred embodiment the compositions are formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The neuraminidase inhibitors and or immunomodulators may be chemically modified so that oral delivery of the derivative is efficacious. Generally, the chemical modification contemplated is the attachment of at least one moiety to the component molecule itself, where the moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The neuradimindase inhibitors and/or immunomodulators can be incorporated into an inert matrix which permits release by either diffusion or leaching mechanisms, e.g., gums. Slowly degenerating matrices may also be incorporated into the formulation. For oral formulations, the location of release may be the stomach, the small intestine (the duodenum, the jejunem, or the ileum), or the large intestine. Preferably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films. Oral formulations may be in the form of chewing gum, gel strips, tablets or lozenges.

2. Topical or Mucosal Delivery Formulations

Compositions can be applied topically. The compositions can be delivered to the lungs while inhaling and traverses across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent™ nebulizer (Mallinckrodt Inc., St. Louis, Mo.); the Acorn II™ nebulizer (Marquest Medical Products, Englewood, Colo.); the Ventolin™ metered dose inhaler (Glaxo Inc., Research Triangle Park, N.C.); and the Spinhaler™ powder inhaler (Fisons Corp., Bedford, Mass.).

Formulations for administration to the mucosa will typically be spray dried drug particles, which may be incorporated into a tablet, gel, capsule, suspension or emulsion.

Transdermal formulations may also be prepared. These will typically be ointments, lotions, sprays, or patches, all of which can be prepared using standard technology. Transdermal formulations will require the inclusion of penetration enhancers.

3. Controlled Delivery Polymeric Matrices

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of the disclosed compounds, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release 5, 13-22 (1987); Mathiowitz, et al., Reactive Polymers 6, 275-283 (1987); and Mathiowitz, et al., J. Appl. Polymer Sci. 35, 755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### III. Methods of Treatment

It has been discovered that the combination of one or more neuraminadase inhibitors with one or more, preferably two, anti-inflammatory agents can effectively treat influenza H5N1 in subjects infected for at least 24, 48, or even 72 hours. The survivability rates of influenza infected subjects treated with the disclosed triple combination compositions increased compared to treatment with a neuraminidase inhibitor alone. Preferred influenza viruses to be treated include, but are not limited to, influenza A (H5N1).

Infected birds have been the primary source of influenza A (H5N1) infections in humans in Asia. The avian influenza A (H5N1) has virulence factors including the highly cleavable hemagglutinin that can be activated by multiple cellular proteases, a specific substitution in the polymerase basic protein 2 (Glu627Lys) that enhances replication (Hatta, M., et al., Science, 293:1840-1842 (2001); Shinya, K., et al., Virology, 320:258-266 (2004)), a substitution in nonstructural protein 1 (Asp92Glu) that confers increased resistance to inhibition by interferons and tumor necrosis factor (TNF-α) in vitro and prolonged replication in swine, (Seo, S. H., et al., Nat Med, 8:950-954 (2002)), as well as greater elaboration of cytokines, particularly TNF-α, in human macrophages exposed to the virus (Cheung, C. Y., et al., Lancer 360:1831-1837 (2002)). Since 1997, studies of influenza A (H5N1) (Guan, Y., et al., Proc Natl Acad Sci USA; 99:8950-8955 (2002)); Li, K. S., et al. Nature, 430:209-213 (2004); Weekly Epidemiol Rec 79(7):65-70 2004)) indicate that these viruses continue to evolve. Such changes include: changes in antigenicity (Sims, L. D., Avian Dis, 47:Suppl:832-838 (2003); Horimoto, T., et al. J Vet Med Sci; 66:303-305 (2004)) and internal gene constellations; an expanded host range in avian species (Sturm-Ramirez, K. M., et al., J Virol, 78:4892-4901 (2004); Perkins, L. E., et al., Avian Dis, 46:53-63 (2002)); the ability to infect fields (Keawcharoen, J., et al., Emerg Infect Dis, 10:2189-2191 (2004); Thanawongnuwech, R., et al., Emerg Infect Dis, 11:699-701 (2005)); enhanced pathogenicity in experimentally infected mice and ferrets, in which they cause systemic infections (Zitzow, L. A., et al., J Virol, 76:4420-4429 (2002); Govorkova, E. A., et al., J Virol, 79:2191-2198 (2005)); and increased environmental stability.

Phylogenetic analyses indicate that the Z genotype has become dominant (Li, K. S., et al. Nature, 430:209-213 (2004)), and that the virus has evolved into two distinct clades, one encompassing isolates from Cambodia, Laos, Malaysia, Thailand, and Vietnam, and the other isolates from China, Indonesia, Japan, and South Korea. Recently, a separate cluster of isolates has appeared in northern Vietnam and Thailand, which includes variable changes near the receptor-binding site and one fewer arginine residue in the polybasic cleavage site of the hemagglutinin.

The virologic course of human influenza A (H5N1) is incompletely characterized, but studies of hospitalized patients indicate that viral replication is prolonged. In 1997, virus could be detected in nasopharyngeal isolates for a median of 6.5 days (range, 1 to 16). In Thailand, the interval from the onset of illness to the first positive culture ranged from 3 to 16 days. Nasopharyngeal replication is less than in human influenza, (Peiris, J. S., et al., Lancet, 363:617-619 (2004)) and studies of lower respiratory tract replication are needed. The majority of fecal samples tested have been positive for viral RNA (seven of nine), whereas urine samples were negative. The high frequency of diarrhea among affected patients and the detection of viral RNA in fecal samples, including infectious virus in one case, (de Jong, M. D., et al., N Engl J Med, 352:686-691 (2005)) suggest that the virus replicates in the gastrointestinal tract. The findings in one autopsy confirmed this observation (Uiprasertkul, M., et al., Emerg Infect Dis, 11:1036-1041 (2005)).

Highly pathogenic influenza A (H5N1) viruses possess the polybasic amino acid sequence at the hemagglutinin-cleavage site that is associated with visceral dissemination in avian species. Invasive infection has been documented in mammals, (Hatta, M., et al., Science, 293:1840-1842 (2001); Shinya, K., et al. Virology, 320:258-266 (2004); (Zitzow, L. A., et al., J Virol, 76:4420-4429 (2002); Govorkova, E. A., et al., J Virol, 79:2191-2198 (2005)), and in humans, six of six serum specimens were positive for viral RNA four to nine days after the onset of illness. Infectious virus and RNA were detected in blood, cerebrospinal fluid, and feces in one patient (de Jong, M. D., et al., N Engl J Med, 352:686-691 (2005)). Whether feces or blood serves to transmit infection under some circumstances is known.

The disclosed compositions are useful for the treatment of one or more symptoms of a viral infection, preferably influenza infection, most preferably influenza A (H5N1) infection. One embodiment provides a method for treating one or more symptoms of influenza in a subject by administering to the subject an effective amount of a neuraminidase inhibitor in combination with an effective amount of one or more, preferably at least two, immunomodulators. A preferred neuraminidase inhibitor is zanamivir. Preferred immunomodulators include anti-inflammatory agents. Most preferred anti-inflammatory agents include celecoxib and mesalazine. The neuramindase inhibitor and the anti-inflammatory agents can be administered as a unit dose formulation or individually. Typically, the composition is administered within at least 12, 24, 48, or 72 hours post-infection.

For all of the disclosed compounds, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 100 mg/kg of body weight daily are administered to mammals. Exemplary adult oral unit doses include oseltamir: 75 mg/day; celecoxib: 200-400 mg/day; mesalazine: 1000 mg/day; and gemfibroxzil: 1200 mg. For inhalation zanamavir, 2 inhalations (one 5-milligram blister per inhalation) twice a day can be used. It is within the abilities of one of skill in the art to adjust the dose of the drug based on body weight. Generally, for intravenous injection or infusion, dosage may be lower.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### Treatment of Mice with Anti-Viral in Combination with Immunomodulators

Methods and Materials

Animal Model and Viral Challenge.

BALB/c female mice, 5 to 7 weeks old, were purchased from the Laboratory Animal Unit of the University of Hong Kong. Mice were kept in biosafety level 3 housing and given access to standard pellet feed and water ad libitum. Aliquots of stocks of influenza A virus strain A/Vietnam/1194/04 were grown in embryonated eggs. Virus-containing allantoic fluid was harvested and stored in aliquots at −70° C. The 50% lethal dose (LD₅₀) was determined in mice after serial dilution of the stock. One thousand LD₅₀ was used for viral challenge in all the experiments. Influenza virus infection was established by intranasal inoculation of mice anesthetized by isoflurane.

Antiviral and Immunomodulatory Treatments.

Antiviral and immunomodulators were administered by the intraperitoneal (i.p.) route using 0.5 ml 29 gauge ultrafine needle insulin syringes. The administered dosage for each agent followed protocols previously described (Budd, A, et al., Antimicrob Agents Chemother 51:2965-2968 (2007); Smith, P. W., et al., J Med Chem 41:787-797 (1998); Ryan, D. M., et al., Antimicrob Agents Chemother 38:2270-2275 (1994); Catalano, A., et al., Int J Cancer 109:322-328 (2004); Sudheer, Kumar M., et al. Mutat Res 527:7-14 (2003)). Control mice were given phosphate buffered saline (PBS) i.p. on the same days (Table 1). Survival, body weight and general conditions were monitored for 21 days or till death.

Experiments were conducted in duplicates or triplicates of 5 mice for each group of treated or control mice. Six mice in each of the groups (groups 8, 11 and 12 in Table 1) were sacrificed on day 4, 6 and 8 post-challenge, respectively. Blood, tracheal-pulmonary lavage, lung, brain, kidney, liver and spleen tissue samples were collected from these mice, normal uninfected mice, and the survived mice for histopathological, immunological and virological assays.

Statistical Analysis.

Statistical analysis of survival time and rate were performed by the log rank Kaplan-Meier and chi square tests respectively, while the others were calculated by Student's t test using Stata statistical software. Results were considered significant at P≦0.05. The Cox proportional hazards model was used to estimate hazard ratios.

Results

Although oseltamivir is highly effective in mouse models, the case-fatality rate remains very high in humans and delayed initiation of therapy appeared to have a detrimental effect on survival. Many antiviral treatment studies of mouse models infected by influenza A/H5N1 virus used an inoculum of about 10 LD₅₀. Good treatment results were obtained if the antiviral was started 4 hours before, soon after or within 36 hours after inoculation (Leneva, I. A., et al., Antiviral Res 48:101-115 (2000); Govorkova, E. A., et al., Antimicrob Agents Chemother. 45:2723-2732 (2001)). Only a few studies showed good results even when the antiviral was started after 36 hours. However, in these series, either a low viral inoculum was used or a duck H5N1 virus adapted to mice was used instead of a human virus for inoculation (Yen, H. L., et al., J Infect Dis, 192:665-672 (2005); Sidwell. R. W., et al., Antimicrob Agents Chemother, 51:845-851 (2007); Simmons, C. P., et al, PLoS Med, 4:e178 (2007)). Thus the pathophysiological status of the infected mice in these studies could be quite different from the real clinical situation when patients often did not present to the hospital till two to four days after the onset of symptoms and the viral load in respiratory secretions was high. The high inoculum and delayed therapy in the presently reported mouse model provided a more realistic simulation for testing various forms of therapy. To avoid the confounding effects of poor oral bioavailability of oseltamivir in sick mice and the known risk of emergence of oseltamivir resistance during therapy, intraperitoneal zanamivir was used.

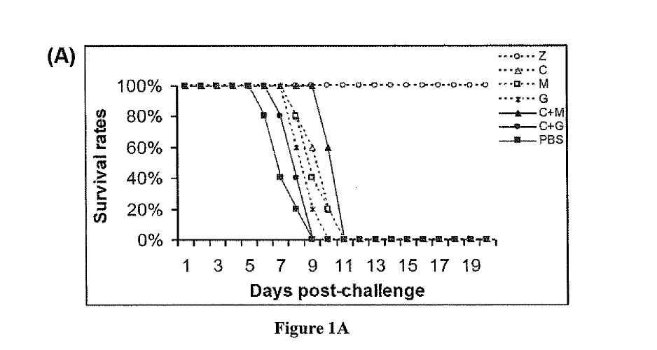

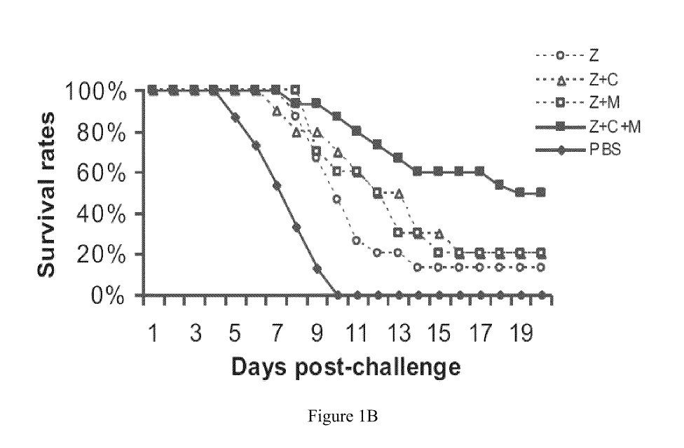

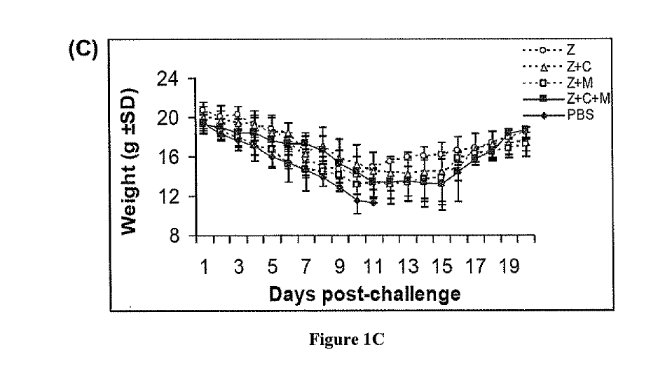

All mice survived with early institution of intraperitoneal (i.p.) zanamivir treatment (FIG. 1A). The survival rate of mice was decreased to 13.3% (2/15) if the treatment with zanamivir was delayed for 48 hours though the mean survival time was prolonged to 10.7±1.6 days compared with 6.6±1.6 days in the controls (FIG. 1B). This provided an ideal situation for testing combination therapy with immunomodulators which had no antiviral effects or any significant effect on survival if used alone.

All PBS-treated controls died. All mice on immunomodulators alone died, but with a trend towards increased mean survival time to about 8.5 days for mice given celecoxib or mesalazine and about 9.5 days for those given both celecoxib and mesalazine, but only about 7.5 days for those given gemfibrozil alone or both celecoxib and gemfibrozil. Therefore, gemfibrozil was not selected for further study. Single use of any of these immunomodulators did not confer survival benefit. However, when zanamivir was combined with both of these two immunomodulators, the survival rate increased to 53.3% (8/15) (P=0.02) and the mean survival time increased to 13.3 days (P=0.0179) compared to zanamivir alone (survival rate 13.3% and survival time 8.4 days). The body weight of all infected mice steadily decreased to a minimum at day 11 and then increased again for those which survived (FIG. 1C).

TABLE 1
Treatment regimens containing zanamivir, celecoxib, mesalazine
and gemfibrozil used alone or in combination for infected mice.

| Groups | Treatment regimens | Numbers |
|---|---|---|
| 1 | 3 mg zanamivir in PBS | 5 |
| | i.p. once every 12 h × 8 | |
| | days* | |
| 2 | 2 mg celecoxib in 10% | 5 |
| | DMSO/PBS i.p. once per | |
| | day × 8 days* | |
| 3 | 1 mg mesalazine in | 5 |
| | ddH2O i.p once per | |
| | day × 8 days* | |
| 4 | 1 mg gemfibrozil in | 5 |
| | propylene glycol i.p. | |
| | once per day × 8 days* | |
| 5 | 2 mg celecoxib + 1 mg | 5 |
| | mesalazine i.p. once per | |
| | day × 8 days* | |
| 6 | 2 mg celecoxib + 1 mg | 5 |
| | gemfibrozil once per | |
| | day × 8 days* | |
| 7 | PBS i.p. once per day × | 5 |
| | 8 days* | |
| 8 | 3 mg zanamivir once | 33^{§} |
| | every 12 hours × 6 days^{†} | |
| 9 | 3 mg zanamivir + 2 mg | 10 |
| | celecoxib i.p. × 6 days^{†} | |
| 10 | 3 mg zanamivir + 1 mg | 10 |
| | mesalazine i.p. × 6 days^{†} | |
| 11 | 3 mg zanamivir + 2 mg | 33^{§} |
| | celecoxib + 1 mg | |
| | mesalazine i.p. × 6 days^{†} | |
| 12 | PBS i.p. once per day × | 33^{§} |
| | 6 days | |

BALB/c mice (female, aged 5-7 weeks) were intranasally challenged with 1,000 LD50 of H5N1 virus strain A/Vietnam/1194/04.
*The treatments started 4 hours post-challenge.
^{†}The treatments started 2 days post-challenge.
^{§}Experiments were conducted in triplicates of 5 mice for each group.

Furthermore, six mice in each of these groups were sacrificed on day 4, 6 and 8 post-challenge, while all survived mice were sacrificed on day 21 post-challenge. Blood, tracheal-pulmonary lavage, lung, brain, kidney, liver and spleen were collected from these mice.

### Example 2

### Decrease in Viral Titers

Materials and Methods

Virological Tests.

Titers of released virus in tracheal-pulmonary lavage were determined by TCID₅₀, while the intracellular viral RNA in lung tissues was quantified by real-time RT-PCR (Li, B. J., et al. Nat Med 11:944-951 (2005); Zheng, B. J., et al. Antivir Ther 10: 393-403 (2005); Wang, M., et al., Emerg Infect Dis 12:1773 1775 (2006)). Briefly, total RNA in lysed lung tissues was extracted using RNeasy Mini kit (Qiagen, Germany) and reverse transcribed to cDNA using applied SuperScript II Reverse Transcriptase™ (Invitrogen, USA). Viral NP gene and internal control-actin gene were measured by SYBR green Mx3000 Real-Time PCR System (Stratagene, USA), using primers NP-Forward: 5′-GAC CAG GAG TGG AGG AAA CA-3′ (SEQ ID NO:1), NP-Reverse: 5′-CGG CCA TAA TGG TCA CTC TT-3′ (SEQ ID NO:2); -Actin-Forward: 5′-CGT ACC ACT GGC ATC GTG AT-5′ (SEQ ID NO:3), -Actin-Reverse: 5′-GTG TTG GCG TAC AGG TCT TTG-3′ (SEQ ID NO:4).

ELISA.

Pro-inflammatory cytokines and chemokines IL-1, IL-6, IFN-γ, TNF-α (BD Biosciences, USA), prostaglandin E2 (PGE2), macrophage inflammatory protein 1 (MIP-1) (R&D Systems Inc, USA), leukotriene (GE Healthcare, UK) and lung injury indicator albumin (BETHYL Laboratories Inc., USA) in tracheal-pulmonary lavage and serum samples were tested by ELISA using the protocol described previously (Zheng, B. J., et al., Vaccine 19:4219-4225 (2001); Zheng, B. J., et al., Eur J Immun 32:3294-3304 (2002)) with modifications according to the instructions of the kits suppliers.

Elastase Activity Assay.

Elastase activity in tracheal-pulmonary lavage was measured by the addition of the elastase-specific chromogenic substrate N-methoxysuccinyl-Ala-Ala-Pro-Val p-nitroanilide (SEQ ID NO:5) (Sigma, USA) at a final concentration of 1 mM. After 30 minutes at room temperature, the change in optical density at a wavelength of 405 nm was measured.

Results

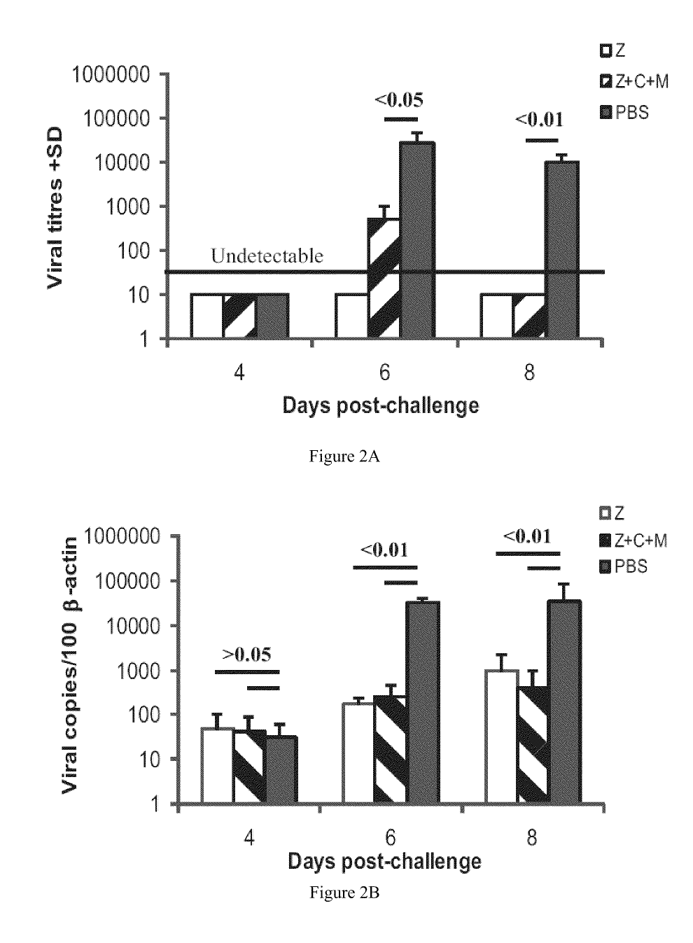

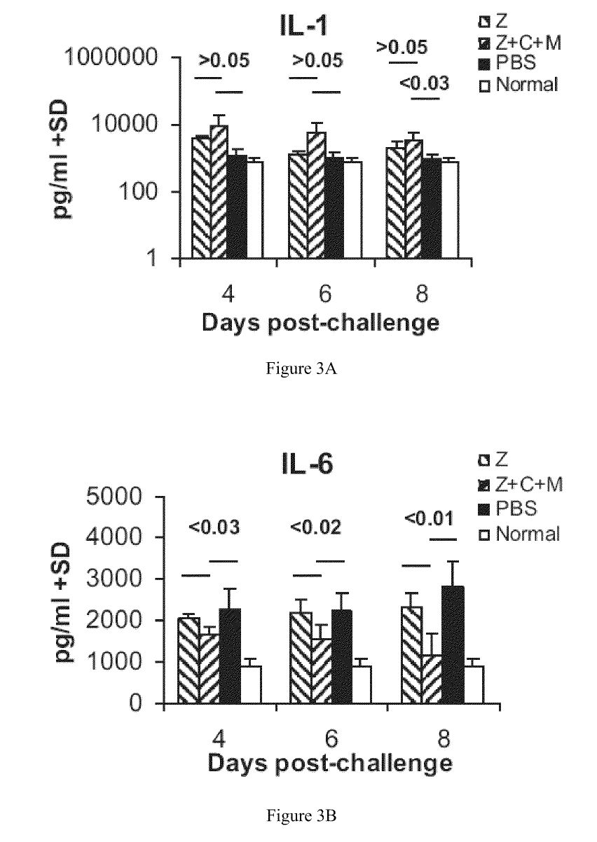

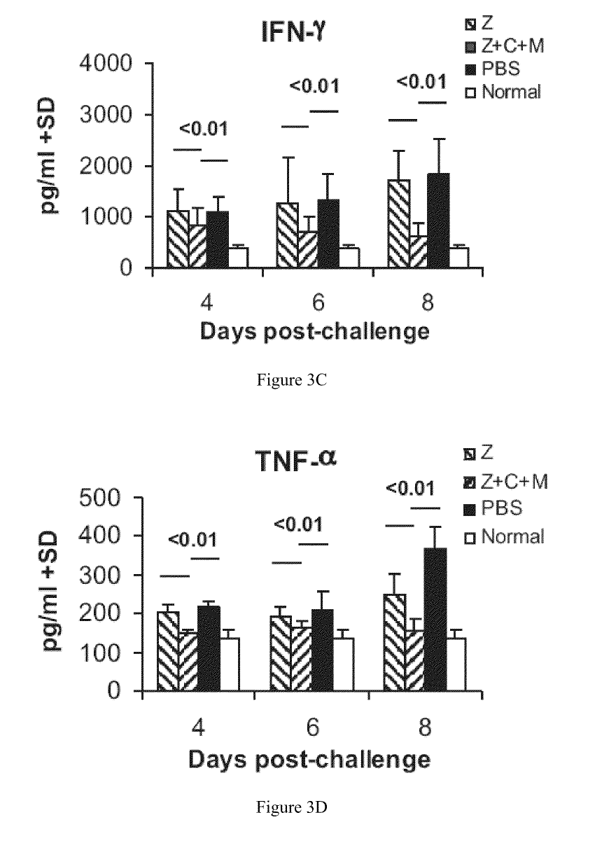

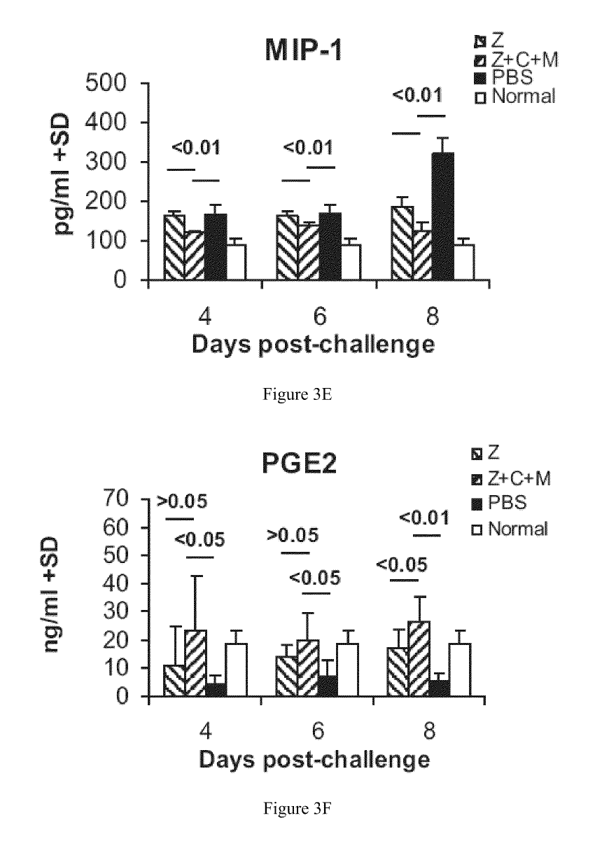

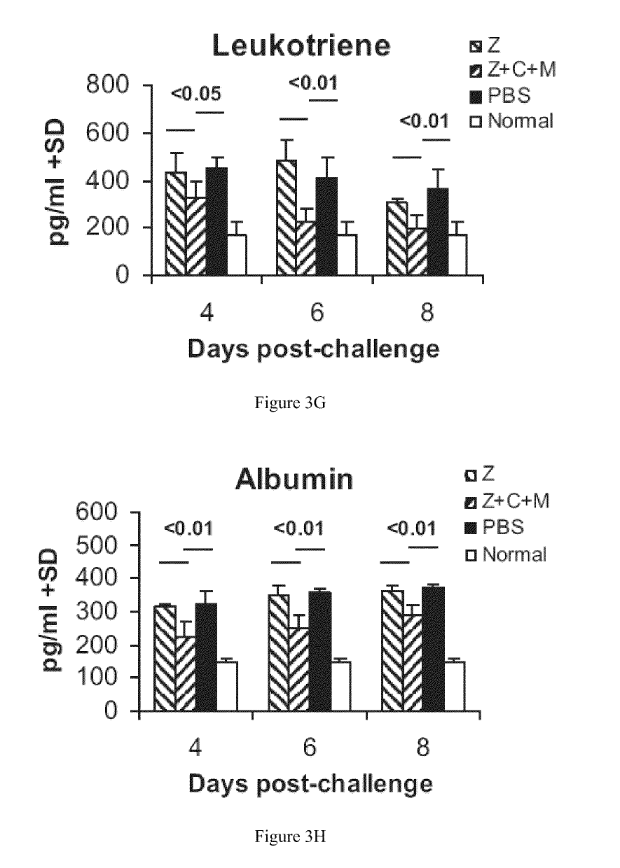

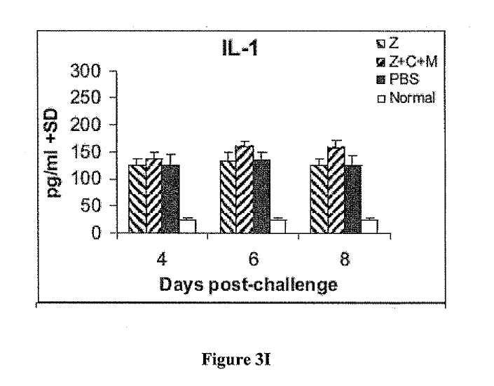

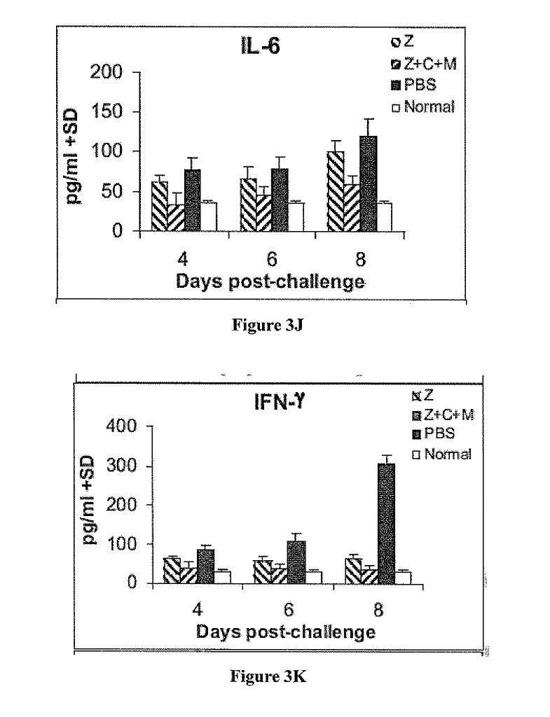

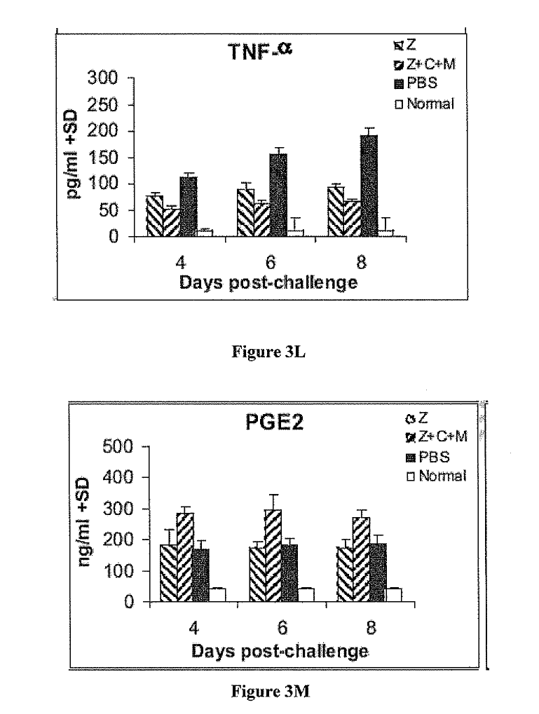

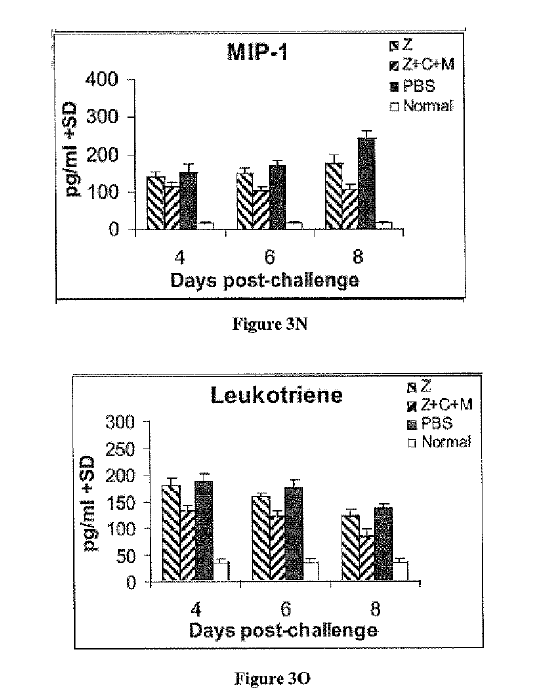

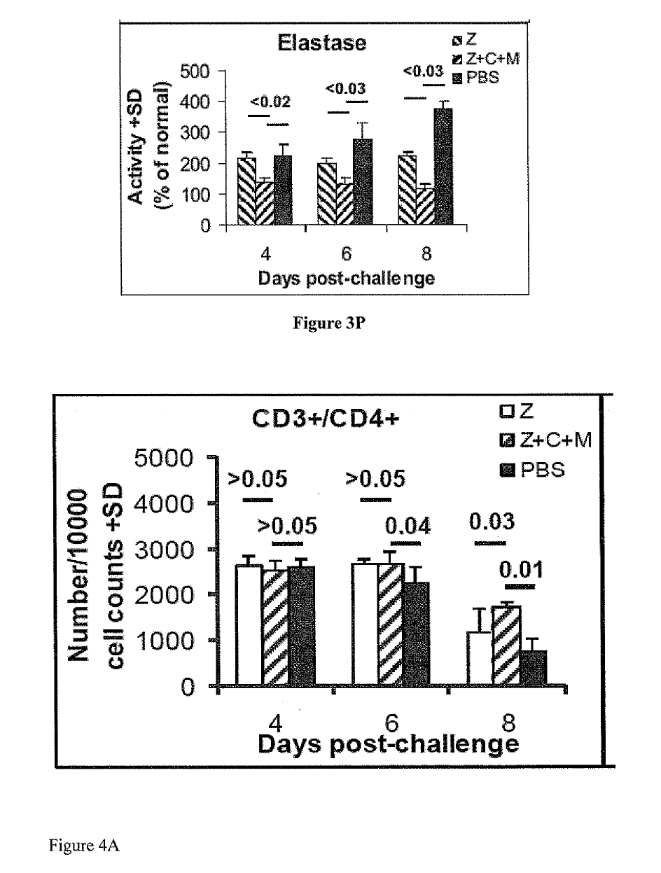

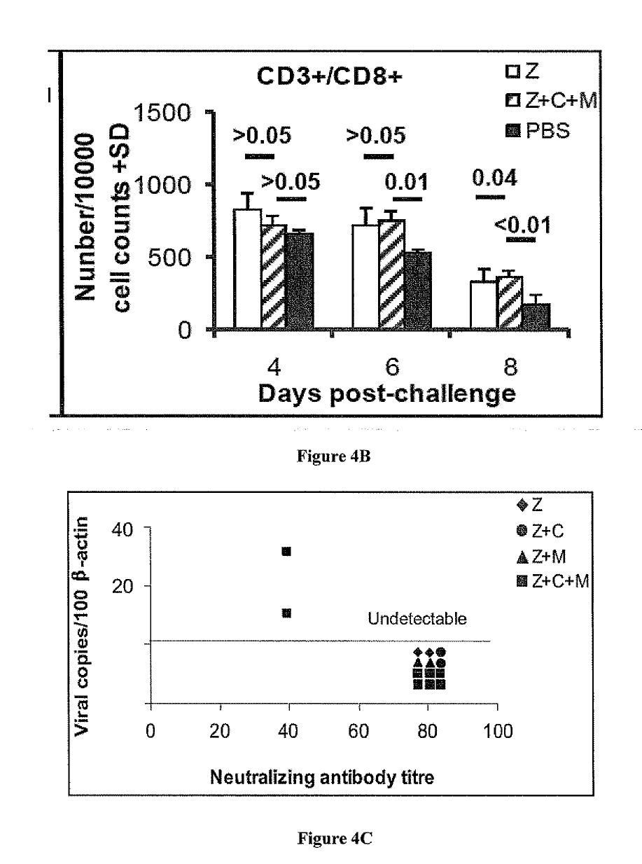

Significant decreases (>2.5 logs) of viral titers in tracheal-pulmonary lavage by TCID₅₀ or copies of viral RNA genomes in lung tissues by real-time quantitative RT-PCR was found in groups treated by zanamivir with or without immunomodulators at day 6 and 8 post-challenge (FIGS. 2A and B). Levels of inflammatory markers IL-6, IFN-γ, TNF-α, MIP-1 and leukotriene assayed by enzyme immunoassays were significantly higher in tracheal-pulmonary lavage obtained from the mice treated with zanamivir alone and controls than those treated by triple therapy (P<0.01 or 0.05) or uninfected normal mice (FIGS. 3A-G). However, IL-1 levels were only slightly lower in those treated with zanamivir alone and controls (P>0.05), while PGE2 levels were found to be significantly higher in the samples collected at day 8 post-challenge from the group receiving triple therapy (FIG. 3F). As expected, their serum cytokine and chemokine changes were similar to those of their tracheal-pulmonary lavage (FIGS. 3H-P) Furthermore, levels of both CD4+ and CD8+ T lymphocytes were significantly higher in the blood taken at day 6 and/or 8 from the mice given triple therapy than those taken from zanamivir-treated and PBS control mice (FIGS. 4A-B). As expected, the degree of lung damage as evident by the albumin concentration (FIG. 3G) and elastase activity (FIG. 3P) in tracheal-pulmonary lavage was significantly lower in groups treated by the combination of antiviral and immunomodulators when compared with the group treated by zanamivir alone (P<0.01) or PBS control (P<0.03).

### Example 3

### Histology

Materials and Methods

Histopathological Analysis.

The lung, brain, spleen, kidney and liver tissues of challenged mice were immediately fixed in 10% buffered formalin and embedded in paraffin wax. Sections 4-6 μm in thickness were mounted on slides. Histopathological changes were examined by hematoxylin and eosin (H&E) staining under light microscope as described by Zheng, B. J., et al., Eur J Immun 32:3294-3304 (2002); Zheng, B. J., et al. Int J Cancer 92: 421-425 (2001).

Immunohistochemical Assay.

Lung sections were stained as described previously (28, 30) using an anti-influenza nucleoprotein monoclonal antibody (HB65, ATCC, USA) at 1:5000 dilution, goat anti-mouse IgG, H & L chain specific biotin conjugate (Calbiochem, USA) at 1:2000 dilution and streptavidin/peroxidase complex reagent (Vector Laboratories, USA).

Flow Cytometry.

Blood cells from the mice were stained with fluorescein-labelled monoclonal antibodies specific for mouse CD3, CD4 and CD8 (BD Pharmingen, USA) and fixed with 4% p-formaldehyde overnight. The fixed blood cells were analyzed by flow cytometry (FACSCaliber, BD, USA) as described previously (Zheng, B. J., et al. J Viral Hepat 11:217-224 (2004)).

Results

Histopathological examination showed that the alveolar damage and interstitial inflammatory infiltration in mice treated by the combination were much less severe than those treated by zanamivir alone (FIG. 4B). There was focal mild perivascular mononuclear cell infiltration in the cerebral cortex from the mice treated with zanamivir alone but not in those from mice treated by both zanamivir and immunomodulators, while focal dense mononuclear cell infiltration in the cerebral cortex was observed in brain tissues taken from the untreated mice. Reactive lymphoid cells which appeared paler in staining were found in spleens obtained from zanamivir-treated and PBS control mice, in which reactive lymphoid cells were present along with frequent apoptotic bodies with prominent nuclear fragmentation, but not in those collected from mice treated with zanamivir and immunomodulators. Nevertheless, no significant pathological changes or tissue damages could be detected in liver and kidney from all mice.

### Example 4

### Presence of Neutralizing Antibodies in Treated Mice

Materials and Methods

Neutralization Assay.

Neutralizing antibody levels in serum samples of the mice were determined by neutralization assay using the same virus strain for challenge in MDCK cells as described by Peiris, J. S., et al., Lancer 363:617-669 (2004), Wang, M, et al., Emerg Infect Dis 12:1773-1775 (2006).

Western Blot.

Influenza A viral proteins NP from H5N1 strain A/Indonesia/5/2005, HA1 from H5N1 strain A/Vietnam/1203/2004 (Immune Technology, USA), HA2 from strain A/Vietnam/1194/04 which was expressed in baculovirus vector (BD Bioscience) were separated in 12% SDS-PAGE gel and then electroblotted onto polyvinylidine difluoride membrane. The membranes were incubated with mouse sera at 1/200 dilution, washed and then incubated with HRP-conjugated anti-mouse IgG monoclonal antibody at a dilution of 1/1000 (Abeam, USA). The blots were detected by the ECL Western blotting detection system (Amersham Biosciences, USA).

Results

As shown in FIG. 4C, 12 surviving mice with undetectable viral load in lung tissues at day 21 after viral challenge also had a neutralizing antibody titer of 80. Western blot confirmed that the neutralizing antibody reacted specifically with baculovirus-expressed proteins of nucleoprotein and hemagglutinin of influenza A/H5N1 virus. Two surviving mice treated with triple therapy still had a detectable low viral load and a neutralizing antibody titer of 40. Compared with the zanamivir-treated group whose TCID₅₀ titer in the tracheal-pulmonary lavage was below our detectable limit, the triple therapy group had a TCID₅₀ titer of 5.1×10²±4.9 102 which was still 2.5 log below the titer of 2.7×10⁵±2.0×10⁵ in the PBS control group (FIGS. 2A-B). The immunomodulators may have some degree of immunosuppression which is not clinically apparent. Consistent with these findings, these two mice [Z+C+M(2)], together with the survived mouse from zanamivir-treated group (Z), also had inflammatory infiltrate in their alveoli on histological examination, whereas no significant inflammation was observed in the other surviving mice [Z+C, Z+M and Z+C+M(6)], which was similar to those found in normal mice.

This study showed that even if the viral replication had been suppressed in the mice treated with antiviral, levels of cytokines and chemokines were still similar to the untreated mice, which were significantly higher than those in the mice receiving combination therapy. This suggests that once the viral infection has triggered the cytokine storm, even if viral replication is suppressed by antiviral therapy, the pro-inflammatory cytokines and chemokines will continue to drive the immunopathological progression, which may explain why antiviral therapy alone may not be clinically effective if the commencement of treatment is delayed. Previous studies showed that anti-inflammatory dose of steroid was not useful in mice (Salomon, R., et al., Proc Natl Acad Sci USA, 104:12479-12481 (2007)) and was associated with significant side effects in human infected by the H5N1 virus without improving the survival (Carter, M. J., J Med Microbiol, 56:875-883 (2007)). Therefore other immunomodulators have to be considered. Ideally, the choice of agents should be targeted to the abnormalities in the immune response to the infection.

First, severe or fatal infections are associated with disseminated viral replication in the body and high viral loads were detected (de Jong, M. D., et al., Nat Med, 12:1203-1207 (2006)). In this regard, antiviral treatment is a crucial aspect of therapy. Secondly, the extensive uncontrolled viral multiplication drives a “cytokine storm” with markedly elevated levels of inflammatory cytokines in blood and from alveolar and bronchial epithelial cells in vitro. These include IP-10, interferon-γ, interferon-β, RANTES, IL-6, IL-8, IL-10, MIP-1, and MCP-1 (Peiris, J. S., et al., Lancet, 363:617-669 (2004); de Jong, M. D., et al., Nat Med, 12:1203-1207 (2006)). Thirdly, apoptosis, especially in pulmonary alveoli and lymphoid tissues leading to lymphopenia, appears to be a prominent pathological feature in patients who died from influenza A/H5N1 infection (Uiprasertkul, M., et al., Emerg Infect Dis, 13:708-712 (2007)). Immunomodulators directed to mitigate the effects of cytokine dysregulation and apoptosis may therefore relieve the morbidity and mortality of the host in the presence of an effective antiviral coverage.

Since COX-2 knockout mice had significantly better survival after challenge with mouse adapted influenza A H3N2 virus than wild type BALB/c mice (Carey, M. A., et al., J Immunol, 175:6878-6884 (2005)), intraperitoneal celecoxib was chosen in this study. Sulfasalazine and related compounds such as mesalazine and 5-aminosalicylic acid are were also chosen in this study because they are highly active in alimentary tract epithelial cells and are commonly used in the treatment of inflammatory bowel diseases. They have diverse effects on the immune system including inhibition of lipoxygenase (LPO) and cyclooxygenase (COX) pathways, which decreases pro-inflammatory cytokines and eicosanoids, and therefore decreases the activation of inflammatory cells such as macrophages and neutrophils. Many of these functions are shared with non-steroidal anti-inflammatory agents. In addition, sulfasalazine and 5-aminosalicylic acid inhibits NF-κB activation and promote the synthesis of phosphatidic acid, both of these actions inhibit the action of ceramides which are potent stimulators of apoptosis (Nielsen, O. H., et al., Nat Clin Pract Gastroenterol Hepatol, 4:160-170 (2007); Gómez-Muñoz, A., et al., Biochim Biophys Acta, 1533:110-118 (2001)). It is likely that the combined actions of mesalzaine (the effective moiety of sulfasalazine) and celecoxib has a synergistic effect to protect the host from excessive damage from cytokine dysregulation and apoptosis following influenza A/H5N1 infection. Both celecoxib and mesalazine are relatively inexpensive, currently used in humans, not known to cause immunosuppression, and relatively free from adverse drug interactions or major side effects with short-term use.

The main target of action of the fibrates such as gemfibrozil is the peroxisome proliferators-activated receptors alpha (PPARα). PPAR are members of the nuclear receptor superfamily which affects the lipid and glucose metabolism, as well as modulation of inflammatory responses. PPAR-α and -γ ligands possess anti-inflammatory activities. PPARα activation is associated with inhibition of NF-KB, COX-2 activity, and production of pro-inflammatory cytokines such as IL-6 and TNF-α (Chinetti, G., et al., Inflamm Res, 49:497-505 (2000)). Therefore, activation of the PPARα by gemfibrozil can be expected to damp down the excessive inflammatory response. Budd et al. demonstrated that gemfibrozil improved survival of mice infected by influenza A/H2N2 virus from 26% (controls) to 52% (treated) (Budd, A., et al., Antimicrob Agents Chemother, 51:2965-2968 (2007)). However, no significant improvement on survival was noted when the hypervirulent H5N1 virus was used in this study. The lack of beneficial effects of gemfibrozil alone in our study could be related to the different pathophysiology between H2N2 and H5N1 viruses or the relatively weak agonistic activities of gemfibrozil on PPARα.

The association between higher levels of PGE₂ and survival of the animals is compatible with the known immunological profiles of human and experimental influenza A/H5N1 infection. Amongst other cytokines and chemokines, severe H5N1 infections are associated with raised levels of RANTES and MIP-1, the synthesis of both of them are inhibited by PGE₂. Our results also showed a reduction in MIP-1 levels following triple therapy. PGE₂ has anti-inflammatory and anti-apoptotic properties, both of which may play a beneficial role in preventing excessive tissue and cellular damage in this animal model. Indeed, the correlation between COX-1 and COX-2 inhibition, PGE₂ levels, and mice survival has been described by Carey et al. using COX-1^{-/-} and COX-2^{-/-} mice infected by influenza A/H3N2 virus (Carey, M. A., et al., J Immunol 175:6878-6884 (2005)). Following infection, COX-2^{-/-} mice had a significantly lower mortality, lesser degree of inflammatory cell infiltrates in the lungs, and lower levels of pro-inflammatory cytokines (TNFα, IL-1, IFN-γ, IL-6) in the tracheal-pulmonary lavage as compared to wild type and/or COX-1^{-/-} mice whereas the PGE₂ levels in the tracheal-pulmonary lavage and the viral load in the lungs were significantly higher in COX-2^{-/-} mice. The findings of lower leukotrienes and higher PGE₂ levels in the tracheal-pulmonary lavage in mice treated by the combination is compatible with the above findings. PGE₂ was shown to be an important lipid mediator which decreases the production of TNF- and other pro-inflammatory cytokines. Though these agents have not been shown to cause immunosuppression, the two mice which survived despite a low level of detectable viral load had received this combination of immunomodulators. The same immunological factors causing tissue damage during the mounting of the immune response may also be critical for viral clearance (La Gruta, N. L., et al., Immunol Cell, 29 Biol 85:85-92 (2007)). IL-1 was speculated to be protective because infected IL-1 receptor knockout mice showed increased morbidity, mortality, lung viral titer and inflammatory infiltrate when infected with a low lethality HK/486 virus (Szretter, K. J., et al., J Virol, 81:2736-2744. (2007)). In this study, mice treated by the combination had improved survival without significant suppression of IL-1 in tracheal-pulmonary lavage despite the use of a hypervirulent virus.

Therefore, the results show the combined use of celecoxib and mesalazine results in a synergistic reduction in the production of pro-inflammatory cytokines, chemokines, and leukotrienes via different pathways. These activities, together with the anti-apoptotic activities of the aminosalicytes, reduce the degree of cell death and tissue damage in the host. The concomitant use of an effective antiviral is essential, not only to limit the extent of viral replication (which drives the cytokine dysfunction) from natural infection, but also to counteract the possible increase in viral load following COX-2 inhibition.

Influenza A/H5N1-infected patients who succumbed often had persistently high levels of serum pro-inflammatory cytokines and chemokines (Peiris, J. S., et al., Lancet, 363:617-669 (2004); de Jong, M. D., et al., Nat Med, 12:1203-1207 (2006)). Therefore, the pathogenesis of the disease was initially attributed to virus-induced cytokine storm. However, studies with knockout mice deficient in TNF, TNFR1, TNFR2, IL-6, CCL2, MIP-1, IL-1R (Salomon, R., et al., Proc Natl Acad Sci USA, 104:12479-12481 (2007); Szretter, K. J., et al., J Virol, 81:2736-2744 (2007)) did not confer better survival after viral challenge when antivirals were not given. Moreover recent studies showed that the levels of serum pro-inflammatory cytokines and chemokines correlated closely with the viral load (de Jong, M. D., et al., Nat Med, 12:1203-1207 (2006)). These reports suggested that the pathogenesis should involve the interplay between a rising viral load and the resulting pro-inflammatory response. Therefore the optimal therapy should consist of both an effective antiviral and immunomodulatory agents especially if the patients present late in the course of the disease when the local and systemic pro-inflammatory cascade are already severely activated.

Post-mortem examination of patients who succumbed to influenza A/H5N1 infection often showed severe lymphopenia and lymphoid atrophy or necrosis in the spleen and other lymphoid tissues (Yuen, K. Y., et al., Lancet, 351:467-471 (1998); Peiris, J. S., et al., Lancet, 363:617-669 (2004)). The study also showed that both CD4+ and CD8+ T lymphocytes were significantly decreased in antiviral treated and untreated mice during disease progression. However, unlike the use of the steroid or other immunosuppressants, the use of celecoxib and mesalazine with zanamivir maintains significantly higher levels of CD4+ and CD8+ T lymphocytes at day 6 and day 8 post-challenge. Histopathological examination also showed that reactive lymphoid cells with frequent apoptotic bodies were found in spleens obtained from zanamivir-treated and untreated mice, but not in those from mice treated with zanamivir and immunomodulators. This suggests that the anti-apoptotic effects of celecoxib plus mesalazine functions to avert the detrimental effects of immunopathological damage.

We claim:

1. A pharmaceutical composition for treating influenza comprising an effective amount of a neuraminidase inhibitor to inhibit or reduce budding of influenza virus from infected cells of a subject and an effective amount of at least two immunomodulators effective to reduce or inhibit one or more symptoms of inflammation of the subject.

2. The pharmaceutical composition of claim 1 wherein the neuraminidase inhibitor is selected from the group consisting of zanamivir, oseltamivir and peramivir.

3. The pharmaceutical composition of claim 2 wherein the neuramindase inhibitor comprises zanamivir.

4. The pharmaceutical composition of claim 1 wherein the immunomodulators are anti-inflammatory agents.

5. The pharmaceutical composition of claim 4 wherein the anti-inflammatory agents are non-steroidal anti-inflammatory agents.

6. The pharmaceutical composition of claim 5 wherein the non-steroidal anti-inflammatory agents are selected from the group consisting of COX-2 inhibitors, aminosalicylate drugs and ligands for PPAR.

7. The pharmaceutical composition of claim 6 wherein the COX-2 inhibitor comprises celecoxib.

8. The pharmaceutical composition of claim 6 wherein the aminosalicylate drug comprises mesalazine.

9. The pharmaceutical composition of claim 1 wherein the neuramindase inhibitor comprises zanamivir, and
wherein the immunomodulators comprise celecoxib and mesalazine.

10. The pharmaceutical composition of claim 1 wherein the influenza is influenza A (H5N1).

11. The pharmaceutical composition of claim 1 wherein the composition extends survivability rates in subjects when administered 24, 48, or 72 hours post infection compared to administration of the neuraminidase inhibitor alone.

12. A unit dose formulation for treating one or more symptoms of influenza comprising an effective amount of zanamivir to inhibit influenza virus from budding from infect cells of a subject and an effective amount of celecoxib and mesalazine to inhibit one or more symptoms of inflammation of the subject.

13. A method for treating influenza comprising administering to subject infected with influenza virus an effective amount of a neuraminidase inhibitor to inhibit or reduce budding of influenza virus from infected cells of the subject and an effective amount of at least two immunomodulators effective to reduce or inhibit one or more symptoms of inflammation of the subject.

14. The method of claim 13 wherein the neuraminidase inhibitor is selected from the group consisting of zanamivir, oseltamivir and peramivir.

15. The method of claim 14 wherein the neuramindase inhibitor comprises zanamivir.

16. The method of claim 13 wherein the immunomodulators are non-steroidal anti-inflammatory agents.

17. The method of claim 16 wherein the non-steroidal anti-inflammatory agents are selected from the group consisting of COX-2 inhibitors, aminosalicylate drugs and ligands for PPAR.

18. The method of claim 13 wherein the neuramindase inhibitor comprises zanamivir, and
wherein the immunomodulators comprise celecoxib and mesalazine.

19. The method of claim 13 wherein the influenza is due to influenza A (H5N1) infection.

20. The method of claim 13 wherein administration at 24, 48, or 72 hours post infection compared to administration of the neuraminidase inhibitor alone increase survivability of the subject compared to administration of the neuraminidase inhibitor alone.
